(19) United States
(12) Patent Application Publication
(10) Pub. No.: US 2008/0287480 A1
(43) Pub. Date: Nov. 20, 2008

(54) Therapeutic Combination Comprising a Nmda Receptors Blocker and a Narcotic Analgesic Substance

(75) Inventors: Paolo Chiesi, Parma (IT); Gino Villetti, Parma (IT)

Correspondence Address: OBLON, SPIVAK, MCCLELLAND MAIER & NEUSTADT, P.C., 1940 DUKE STREET, ALEXANDRIA, VA, 22314, US

(73) Assignee: Chiesi Farmaceutici S.p.A., Parma (IT)

(21) Appl. No.: 11/995,983

(22) PCT Filed: Jul. 18, 2006
(86) PCT No.: PCT/EP2006/007049

§ 371 Date: Jan. 17, 2008

(30) Foreign Application Priority Data
Jul. 28, 2005 (EP) 05016391.4

(51) Int. Cl. A61K 31/485; A61K 31/164; A61P 29/00; A61K 31/445

(52) U.S. Cl. 514/282; 514/619; 514/329

(57) ABSTRACT

The invention relates to a medicament comprising a combination of a substance that blocks both the ion channel associated to NMDA receptors and MAO enzymes and a narcotic analgesic substance, preferably a fixed combination, pharmaceutical compositions thereof, and to the use thereof for the treatment of subjects suffering of various types of pain and/or for inhibiting the development of tolerance, and/or physical dependence on a narcotic analgesic substance.

The present invention relates to a medicament for the simultaneous administration of a substance that blocks both the ion channel associated to NMDA receptors and MAO enzymes, and a narcotic analgesic substance.

In particular, the invention is directed to a medicament in the form of a fixed combination of the two active substances.

The invention also relates to pharmaceutical compositions thereof and to a combination pack or kit containing a combination of the two active substances in distinct dosage forms included in a unique packaging.

The invention further relates to the use of the combination for the treatment of subjects suffering of moderate to severe pain, acute or chronic, whether the pain is of neuropathic or inflammatory type or caused by different nociceptive stimuli, and also mixed pain conditions characterized by the presence of both the acute and chronic components. The combination of the invention can also be used in neuropathic pain states refractory to the treatment with narcotic analgesic substances and/or for inhibiting the development of tolerance, preferably non-associative tolerance, and/or physical dependence on a narcotic analgesic substance.

### BACKGROUND OF THE INVENTION

Pain has been described as an unpleasant sensation that occurs as a result of injury to the body or as a manifestation of a disease state.

Pain can be classified in many ways: on the basis of its duration as acute or chronic pain; by its severity as mild, moderate or severe; by its underlying cause as nociceptive, inflammatory or neuropathic pain.

Acute pain characteristically is of recent onset with a relatively short duration, lasting no more than days or weeks. Acute pain is seen with trauma, surgical interventions and pain caused by certain diseases such as cancerous tumor that invades and stretches an organ.

Chronic pain is defined by the experts as pain persisting for more than 1 month beyond the usual course of an acute illness or the time required for an injury to heal, pain associated with a chronic pathologic process, or pain recurring at intervals of months or years.

Nociceptive pain results from a direct tissue damage deriving for example from surgical incisions, bone fractures, metastatic cancer or joint diseases such as osteoarthritis and rheumatoid arthritis.

Inflammatory pain involves the release of mediators which sensitize peripheral nociceptors. Nociceptors sensitization plays an important role in central sensitization and clinical pain states such as: i) an increased response to a noxious stimulation (hyperalgesia) and ii) a painful response to a normally innocuous stimulus (allodynia).

Neuropathic pain occurs as a result of damage to, or dysfunction of, the nervous system.

Inflammation and neuropathy are the two major pathophysiological changes that active different chronic pain mechanisms.

Chronic inflammatory pain results from peripheral tissue injury produced by infection or trauma such as gout, or diseases with an autoimmune component, such as arthritis.

Chronic neuropathic or neurogenic pain results from nerve injury associated with trauma, radiation damage, surgery, crushed limbs or amputation, and diseases such as herpes zoster, multiple sclerosis, arthritis, and diabetes, or from cancer chemotherapy. Diabetic neuropathy is the most common neuropathic pain syndrome.

Narcotic analgesic substances, i.e. opioids and their derivatives such as fentanyl, hydrocodone, hydromorphone, meperidine, morphine, oxymorphone, phentazocine and tramadol, or pharmaceutically acceptable salts thereof, are widely applied and their efficacy is recognized in managing moderate to severe acute pain.

Morphine is one of the most widely utilized. It is preferably administered orally, and doses should be given at regular intervals to provide good pain control.

When oral morphine is ineffective, the next option is the parenteral administration, preferably intravenously (i.v.) or by continuous infusion.

Clinical studies showed that opioids such as morphine could be effective in some patients suffering from neuropathic pain (Portenoy R K et al Pain 1990, 43, 273-286; Rowbotham M C et al Neurology 1991, 41, 1024-1028).

On the other hand, the long term use of narcotic analgesic substances, has been limited due to their negative side effects such as constipation, sedation, respiratory depression, and principally tolerance and physical dependence, which develop rapidly after administration.

In an effort to make narcotic analgesic substances of wider use in the treatment of pain, in particular chronic pain, various kinds of combinations with other active substances have been described in the prior art.

Among them, combinations of narcotic analgesic substances with substances that blocks the N-methyl-D-aspartate (NMDA) receptor have been proposed, since there is evidence that painful responses such as hyperalgesia and allodynia also depend on NMDA receptor-mediated central changes in synaptic excitability.

Functional inhibition of NMDA receptors can be achieved through actions at different recognition sites such as the primary transmitter site (competitive), strychnine-insensitive glycine site (glycine B), polyamine site (NR2B selective) and phencyclidine site located inside the cationic channel.

The substances that block the phencyclidine site located inside the cationic channel are hereinafter referred to as NMDA channel blockers. NMDA channel blockers act in an uncompetitive “use-dependent” manner meaning that they only block the channel in the open state.

Typical uncompetitive NMDA channel blockers are morphinans such as dextromethorphan or dextrorphan, MK-801, ketamine, memantine and neramexane.

Hereinafter the terms blockers and antagonists are used as synonyms.

The capability of NMDA receptor blockers such as morphinans of reducing or inhibiting tolerance and/or dependence to narcotic analgesic substances in different models of pain has been reported in several documents of the prior art (U.S. Pat. No. 5,321,012; Trujillo et al in Science 1991, 251, 85-87; Ben-Eliyahu S et al Brain Res 575, 304, 1992; Trujillo K et al (Brain Res 1994, 633, 178-188).

More recently, in a review directed to NMDA receptors as targets for drug action in neuropathic pain (Parson C G et al Eur J Pharmacol 2001, 429, 71-78), the authors stated that the antinociceptive effects of NMDA receptor blockers and opioids could be predicted to be synergistic and the presence of an NMDA receptor blocker, besides inhibiting the development of tolerance to the analgesic effects of morphine should block the development of chronic pain states.

For example, Kauppila T et al (Neuroreport 1998 9, 1071-1074) reported on the analgesic effects of 2 mg/kg morphine and 45 mg/kg dextrometorphan, and their combination, after subcutaneous (s.c.) administration, in a different rat model of chronic pain, i.e. mononeuropathy after irradiation of the sciatic nerve. The authors found that the combination markedly alleviated mechanical and cold allodynia while neither drug produced a significant effect on its own at these doses. However, the synergistic effect was demonstrated with a dose of morphine (2 mg/kg s.c.) much higher than those usually considered useful for therapeutic purposes in non opioid tolerant patients by parenteral administration.

Christensen D et al (Br J Pharmacol 1998, 125, 1641-1650),

Pelissier T et al (Eur J Pharmacol 2003, 477, 23-28) and

U.S. Pat. No. 6,538,008 discloses combinations of other NMDA blockers with a narcotic analgesic substance.

However either the narcotic substance was administered at doses much higher than those usually considered useful for therapeutic purposes in non opioid tolerant patients, or the non-opioid drugs in the combinations turned out to be effective at doses that are not recommendable for therapeutic purposes, in consideration of the concomitant side effects. In particular, no demonstration was given that said combinations would be able of antagonizing both hyperalgesia and allodynia from various type of stimuli at therapeutically acceptable doses.

Finally, in none of the analyzed papers dealing with the synergistic antinociceptive effects, the effects on tolerance were contextually investigated.

Therefore there is still an unmet need for an efficacious and well tolerated analgesic therapy for the treatment of moderate-to severe acute and chronic pain.

In particular there is a need for an analgesic therapy useful for the treatment of neuropathic pain, more in particular neuropathic pain states refractory to the treatment with an analgesic narcotic substance. The treatment of pain, in particular neuropathic pain is a clinical challenge also because of the high degree of interpatient variability. Neuropathic patients indeed may be confused by the unusual sensations they are experiencing and unable to effectively describe or communicate their symptoms.

Therefore, it would be particularly advantageous to provide a combination comprising an active substance capable of inhibiting tolerance to the narcotic analgesic substance and acting in an additive or synergistic way in a wider as possible type of pain models, antagonizing both hyperalgesia and allodynia from various type of stimuli at therapeutically acceptable doses.

Acetamide, 2-[(2,3-dihydro-1H-inden-2-yl)amino]monohydrochloride or N-(2-indanyl)-glycinamide mono hydrochloride also referred to as CHF 3381, has been disclosed for the first time in WO 98/03472, for the treatment of chronic neurodegenerative diseases, such as Alzheimer's disease, various forms of dementia, Parkinson's disease, Huntington's disease or acute neurodegenerative impairments such as stroke and head injuries and for the treatment of epilepsy and depression.

CHF 3381 has been then characterized as an uncompetitive NMDA channel blocker and its anticonvulsivant and neuroprotective properties were further investigated.

In WO 03/053429 it was additionally disclosed that CHF 3381 exhibits a unique dual inhibiting activity towards MAO (mono amino oxidase) enzymes and ion channel associated to NMDA receptors and, in virtue of such dual action it was reported to possess an analgesic activity in animal models of neuropathic pain, acute pain and formalin-induced inflammatory pain.

In Villetti G et al (J Pharmacol Exp Ther 2003, 306, 804-814) the activity of CHF 3381 in experimental models of inflammatory and neuropathic pain was further investigated. In the paper, the results of tolerance studies in a model of inflammatory hyperalgesia (mouse paw formalin test) were reported.

However the administration of CHF 3381 in combination with morphine and its effect on morphine tolerance was never reported.

It has now been found that a substance endowed with a dual mechanism of action of inhibition of both MAO enzymes and NMDA channel receptors (hereinafter referred to as NMDA/MAO blocker) can be advantageously combined with a narcotic analgesic substance for the treatment of various types of moderate to severe pain, acute or chronic, and also in mixed pain conditions characterised by the presence of both the acute and chronic components.

In said combination the NMDA/MAO blocker is able of both inhibiting tolerance and increasing the analgesic effect of the narcotic analgesic substance, administered at doses therapeutically acceptable, by oral route as well.

In particular, it has now been found, and it is the object of the present invention, that CHF 3381 can be advantageously combined with narcotic analgesic substances such as morphine for the treatment of various forms of pain, in particular for the treatment of neuropathic pain, more in particular neuropathic pain states refractory to the treatment with an analgesic narcotic substance.

### SUMMARY OF THE INVENTION

The present invention is directed to a medicament for the simultaneous administration of a substance that blocks both the ion channel associated to NMDA receptors and MAO enzymes (hereinafter NMDA/MAO blocker) and a narcotic analgesic substance.

In particular the invention is directed to a medicament comprising a fixed combination a NMDA/MAO blocker and a narcotic analgesic substance.

The invention is also directed to a pharmaceutical composition comprising therapeutically effective amounts of a NMDA/MAO blocker and a narcotic analgesic substance in a unique dosage form, optionally together with at least one pharmaceutically acceptable carrier or diluent.

In a further embodiment, the invention is directed to a combination pack or kit comprising: a) a therapeutically effective amount of a NMDA/MAO blocker in a pharmaceutically acceptable carrier or diluent in a first unit dosage form; b) a therapeutically effective amount of a narcotic analgesic substance or a pharmaceutically acceptable salt thereof in a pharmaceutically acceptable carrier or diluent in a second unit dosage form; and c) a container for containing said first and second dosage forms in a unique packaging.

Moreover, the invention is further directed to the use of a NMDA/MAO blocker in combination with a narcotic substance for the preparation of a medicament for the treatment of subjects suffering of moderate to severe pain, acute or chronic, whether the pain is of neuropathic or inflammatory type or caused by different nociceptive stimuli, and also mixed pain conditions characterised by the presence of both the acute and chronic components. The combination of the invention can also be used in neuropathic pain states refractory to the treatment with narcotic analgesic substances and/or for inhibiting the development of tolerance, and/or physical dependence on a narcotic analgesic substance.

According to a further feature of the invention there is provided a method for the treatment of subjects suffering of moderate to severe pain, acute or chronic, whether the pain is of neuropathic or inflammatory type or caused by different nociceptive stimuli, and also mixed pain conditions characterized by the presence of both the acute and chronic components, said method comprising the administration of a NMDA/MAO blocker in combination with a narcotic analgesic substance.

The therapeutic method of the invention is also effective in neuropathic pain states refractory to the treatment with narcotic analgesic substances and/or for the treatment of subjects which have developed tolerance, preferably non-associative tolerance, and/or physical dependence on a narcotic analgesic substance.

As used herein, the term “fixed combination” means a combination wherein the active substances are present in a fixed amount and quantitative ratio in an unique dosage form.

### DETAILED DESCRIPTION OF THE INVENTION

The present invention is directed to a medicament for the simultaneous administration of a substance that blocks both the ion channel associated to NMDA receptors and MAO enzymes (NMDA/MAO blocker) and a narcotic analgesic substance.

Preferably, the NMDA/MAO blocker is represented by the general formula I:

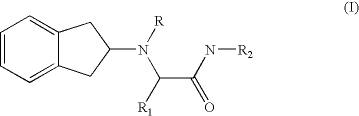

wherein:

R is hydrogen or C₁-C₄ alkyl groups;

R₁ is hydrogen, C₁-C₁₀ alkyl or optionally acylated C₁-C₄ hydroxyalkyl wherein the acylated C₁-C₄ hydroxyalkyl is selected from the group of acetyloxy C₁-C₄ alkyl, propanoyloxy C₁-C₄ alkyl, 2-methylpropanoyloxy C₁-C₄ alkyl, and benzoyloxy C₁-C₄ alkyl;

R₂ is hydrogen; C₁-C₁₀ alkyl; phenyl; phenyl C₁-C₁₀ alkyl; and pharmaceutically acceptable salts thereof.

The preferred NMDA/MAO blocker is a compound of formula (I) wherein R, R₁ and R₂ are hydrogen, also referred to as CHF 3381, more preferably in the form of hydrochloride salt.

It was indeed found that CHF 3381, a representative compound of the pharmacologically class of the NMDA/MAO blockers, combined with morphine, significantly reduced behavioral signs of peripheral neuropathy in an animal model of chronic pain i.e. the chronic constriction injury model of neuropathic pain described by Bennett G J et al in Pain 1988, 33, 87-107.

Said model is considered predictive of chronic pain states refractory to the treatment with narcotic analgesic substances (Mao J et al. Pain 1995, 61, 353-64).

In said model, the simultaneous administration of CHF 3381 and morphine significantly reduced behavioral signs of peripheral neuropathy in a synergistic way. In particular, it was found that the administration of 0.1 mg/kg morphine subcutaneously (s.c.) in combination with 30 mg/kg CHF 3381 per os, was able to significantly reverse both mechanical hyperalgesia as well as cold and mechanical allodynia while neither drug produced a significant effect on its own at these doses.

Surprisingly, morphine turned out to be significantly efficacious in the combination of the invention at a dose of 0.1 mg/kg s.c., a dose which had no or modest effect on its own and which is within the range of the recommended therapeutic doses for parenteral administration in non opioid tolerant patients (0.08-0.2 mg/kg).

In the same model morphine alone significantly reversed hyperalgesia and allodynia at doses ranging from 1 to 3 mg/kg s.c., so from 10 to 30 fold higher.

The combination of CHF3381 and morphine in the above range of doses did not appear to be associated with major side effects.

The combination of CHF3381 and morphine was also tested in a model of inflammatory pain, and it turned out to be capable of preventing or slowing the development of non-associative tolerance induced by morphine in a dose-dependent manner.

As previously reported, NMDA/MAO blockers such as the compounds of formula (I) and in particular CHF 3381 were found to exhibit an analgesic activity in several models of pain.

By virtue of such a broad spectrum of activity and the findings disclosed in the present application, the combination of the invention would turn out to be useful for the treatment of moderate to severe pain, acute or chronic, whether the pain is of neuropathic or inflammatory type or caused by different nociceptive stimuli, and also mixed pain conditions characterized by the presence of both the acute and chronic components.

Advantageously, the NMDA/MAO blocker inhibits competitively and reversibly both the isoforms A and B of MAO enzyme, more advantageously with a more potent action towards MAO-A.

Advantageously, the narcotic analgesic substance is selected from opioids and their derivatives such as alfentanyl, alphaprodine, anileridine, bezitramide, buprenorphine, codeine, dihydrocodeine, diphenoxylate, ethylmorphine, fentanyl, diamorphine (heroin), heptadone, hydrocodone, hydromorphone, isomethadone, levomethorphan, levorphanol, meperidine, metazocine, methadone, metopon, morphine, opium extracts, oxycodone, oxymorphone, pethidine, phentazocine, piminodine, racemethorphan, racemorphan, thebaine, tramadol, or pharmaceutically acceptable salts thereof.

Preferably the narcotic analgesic substance is selected from fentanyl, hydrocodone, hydromorphone, meperidine, morphine, oxymorphone, phentazocine and tramadol, or pharmaceutically acceptable salts thereof such as hydrochloride, sulphate, citrate, lactate and tartrate.

The preferred narcotic analgesic substance is morphine, preferably in the form of hydrochloride or sulphate salt.

Advantageously the medicament is in the form of a fixed combination.

The invention is also directed to a pharmaceutical composition comprising therapeutically effective amounts of a NMDA/MAO blocker and a narcotic analgesic substance in a unique dosage form, optionally together with at least one pharmaceutically acceptable carrier or diluent.

In a further embodiment, the invention is directed to a combination pack or kit comprising: a) a therapeutically effective amount of a NMDA/MAO blocker in a pharmaceutically acceptable carrier or diluent in a first unit dosage form; b) a therapeutically effective amount of a narcotic analgesic substance or a pharmaceutically acceptable salt thereof salt thereof in a pharmaceutically acceptable carrier or diluent in a second unit dosage form; and c) a container for containing said first and second dosage forms in a unique packaging.

For the simultaneous administration of the invention, the two active substances can be administered by any route of administration, for example by oral, intramuscular (i.m.), intravenous (.i.v.), intra-articular, intratechal, epidural, subcutaneous (s.c.), rectal, pulmonary, topical or transdermal route.

The ratios in which the NMDA/MAO blocker and the narcotic analgesic substance may be used in a fixed combination drug are variable depending on the type of the active substance.

Preferably the pharmaceutical composition comprising fixed amounts of the two active substances is administered per os and it can be in the form of tablets, capsules or granules for aqueous suspensions or solutions, more preferably in the form of immediate- or sustained (extended)-release tablets.

The dosage amounts of the NMDA/MAO blocker and the narcotic analgesic substance in said pharmaceutical composition for oral administration can vary depending on the type of the active substance.

In a preferred embodiment, the pharmaceutical composition for oral administration comprises CHF 3381 hydrochloride in an unit dosage amount of 25 mg to 600 mg, preferably 50 mg to 500 mg, more preferably 100 mg to 400 mg.

Advantageously, in said composition, when a narcotic analgesic substance selected from fentanyl, hydromorphone, meperidine, morphine, and tramadol is used, it is present in the following dosage amounts:

fentanyl, from 0.1 mg to 2 mg and preferably 0.2 mg to 1.6 mg, as citrate salt;

hydromorphone, from 2.5 mg to 160 mg and preferably 10 mg to 40 mg, as hydrochloride salt;

meperidine, from 25 mg to 150 mg and preferably 50 mg to 100 mg, as hydrochloride salt;

morphine, from 1 mg to 250 mg, preferably 2.5 mg to 120 mg, more preferably 5 mg to 60 mg, as hydrochloride or sulphate salt;

tramadol, from 25 mg to 250 mg and preferably 50 mg to 200 mg, as hydrochloride salt.

One of the preferred fixed combination drug of the invention comprises CHF 3381 hydrochloride in an unit dosage amount of 100 mg to 400 mg in combination with morphine hydrochloride in an unit dosage amount of 5 mg to 60 mg, preferably 10 mg to 30 mg.

When the simultaneous administration of the two active substances of the combination of the invention is performed by means of a combination kit, in said kit CHF 3381 hydrochloride can be provided in the form of tablets or capsules for oral administration in an unit dosage amount comprised between 20 mg and 600 mg, preferably 50 mg and 500 mg, more preferably between 100 mg and 400 mg.

The analgesic narcotic substance, instead can be provided in the form of tablets or capsules for oral administration or in the form of suppositories for rectal administration or in the form of aqueous solution or lyophilized powders to be reconstituted with water for epidural, intratechal, i.m, i.v. or s.c. administration. Advantageously, when a narcotic analgesic substance selected from hydromorphone, morphine, oxymorphone, penthazocine is used, it is provided in the form of aqueous solution in the following concentrations:

hydromorphone, from 0.5 mg/ml to 5 mg/ml and preferably 1 mg/ml to 4 mg/ml as hydrochloride salt;

morphine, from 0.5 mg/ml to 50 mg/ml mg and preferably 1 mg/ml to 20 mg/ml, as hydrochloride or sulphate salt;

oxymorphone, from 0.5 mg/ml to 2 mg/ml and preferably 1 mg/ml to 1.5 mg/ml, as hydrochloride salt;

penthazocine, from 15 mg/ml to 40 mg/ml, preferably 30 mg/ml as lactate salt.

The medicament of the invention for the simultaneous administration of a NMDA/MAO blocker and a narcotic analgesic substance is indicated for the treatment of subjects suffering of moderate to severe pain, acute or chronic, whether the pain is of neuropathic or inflammatory type or caused by different nociceptive stimuli, and also mixed pain conditions characterized by the presence of both the acute and chronic components.

Examples of moderate to severe acute pain include pain caused by a trauma, or a surgical intervention such as post-operative pain, and pain caused by a disease such as cancer, AIDS, myocardial infarction and renal or biliar colic.

Chronic pain syndromes, comprise a broad clinical group such as chronic inflammatory pain or chronic neuropathic pain. Said syndromes result from peripheral tissue injury produced by infection or trauma or from nerve injury associated with trauma, radiation damage, surgery, crushed limbs or amputation. Chronic pain syndromes also results from diseases such as cancer, nonmalignant progressive disease (e.g., AIDS, sickle cell anemia, hemophilia, and connective tissue diseases), non-progressive or slowly progressive diseases (e.g., severe osteoporosis, gout, post-herpetic neuralgia, painful polyneuropathy, reflex sympathetic dystrophy), and idiopathic syndromes (e.g., fibromyalgia, atypical facial pain, chronic pelvic pain of unknown etiology).

In a particular embodiment of the invention, the combination of the invention is used in the preparation of a medicament for the treatment of subjects suffering of neuropathic pain states refractory to the treatment with narcotic analgesic substances. Examples of said states include for example, the allodynic forms or some forms which occur, in some advanced cancer patients.

In a further embodiment of the invention, the combination of the invention is used in the preparation of a medicament for inhibiting the development of tolerance, preferably non-associative tolerance, and physical dependence on a narcotic analgesic substance, for example in subjects addicted to drugs such as heroin.

Moreover, in comparison to the combinations of the prior art, the combination of the invention would turn out to be safer since NMDA/MAO blockers exhibit a low affinity for the NMDA receptor, producing their analgesic effects in virtue of the specific dual mechanism of action, and do not give rise to psychomimetics effects such as hallucinations and/or cognitive changes on attention and memory which have observed with high affinity NMDA blockers such as ketamine (Fisher K et al J Pain Symptom Management 2000, 20, 358-373; Farber N B Ann N Y Acad Sci, 2003, 1003, 119-130).

The following examples illustrate the invention in greater detail.

### EXAMPLES

### Example 1

### Effect of CHF 3381 and Morphine Combination on Mechanical Allodynia in a Rat Model of Chronic Pain

The animal model of chronic pain was a slight modification of the chronic constriction injury model of neuropathic pain originally described by Bennett G J et al in Pain 1988, 33, 87-107.

Nerve injured rats were placed on an elevated screen in a clear testing chamber and allowed to acclimate to the testing environment before any measurements were taken. The mechanical stimulus was delivered underneath to the plantar surface of the left hind paw of the rat by using the automated testing device Electronic Von Frey.

A steel rod was pushed against the hind paw with ascending force. The ramping of the force went from 0 to 50 g over a 20 s period. When the animal withdrew its hind paw, the mechanical stimulus was automatically withdrawn and the force at which the animal withdrew its paw was recorded. To quantify the mechanical sensitivity of the hind paws, withdrawal thresholds were taken from five consecutive trials with at least 10 s between each trial. The withdrawal threshold was taken to be the mean of the five trials (baseline value). Care was taken to stimulate random locations proximal and distal to the injection site on the plantar surface.

Neuropathic rats were stratified into groups based on their baseline withdrawal thresholds, so that the mean baseline did not differ between groups. The morning before testing, neuropathic rats received three training sessions. Baseline paw withdrawal thresholds were defined as the mean of the last two trials to eliminate the large variability found in the initial withdrawal measurement. To examine the inhibition of nerve injury-induced mechanical hyperalgesia, vehicle, CHF3381 (30 mg/kg p.o.) alone, morphine (0.1 mg/kg s.c.) alone or their combination were administered 60 minutes before mechanical withdrawal thresholds were recorded.

The results are reported and discussed as follows. The values are expressed as Maximum Possible Effect (MPE)±s.e.m

The MPE value was calculated as follow: MPE=(PDR−IBR)/(CBR−IBR), where PDR is the post drug response of the ipsilateral paw, IBR is the ipsilateral paw baseline response and CBR is the contralateral paw baseline response.

The level of significance was set at P<0.05.

Both CHF3381 alone at 30 mg/kg, p.o. and morphine alone at 0.1 mg/kg, s.c. failed to increase the paw withdrawal threshold to mechanical stimulation, being the MPE values 0.01±0.07 and 0.21±0.07 respectively. However, the combination of CHF3381 (30 mg/kg p.o.) with low doses of morphine (0.1 mg/kg s.c) significantly increased paw withdrawal threshold to an MPE value of 0.68±0.06, being the MPE value of vehicle-treated animals 0.15±0.06 (P<0.01).

Therefore the present results show that the combination of CHF 3381 with morphine reduces significantly allodynia after mechanical stimulus in this model of chronic pain at doses which had no effect on their own.

### Example 2

### Effect of CHF3381 and Morphine Combination on cold Allodynia in a Rat Model of Chronic Pain

The animal model of chronic pain was the same of Example 1.

Neuropathic rats were placed upon a metal floor chilled by an underlying water bath (5±1° C.) for a maximum of 20 s. The animals responded to the contact with the cold surface by lifting the paw on the ligated side off the floor. The cold stimulus did not elicit any pain-related paw withdrawal in the sham-operated group. For each set of experiments, animals were pre-screened twice with 20 min interval between tests, in order to select animals displaying clear signs of allodynia, i.e. animals with a paw withdrawal latency on the ligated side of <10 s in both trials. Animals were then stratified into groups based on their mean withdrawal threshold, so that the mean baseline did not differ between groups. The latency to paw withdrawal was then determined at 60 minutes after the administration of vehicle, CHF3381 (30 mg/kg p.o.) alone, morphine (0.1 mg/kg s.c.) alone or their combination.

Mechanical nociceptive thresholds were assessed as reported in the Example 1.

The results, expressed as means ±s.e.m, are reported and discussed as follows. The level of significance was set at P<0.05.

At baseline, nerve injured rats displayed cold allodynia by lifting the ligated hind paw off the floor with mean baseline withdrawal latencies ranging from 3.6±3.78 s. Sixty minutes after treatment, only the co-administration of CHF3381 and morphine significantly increased mean paw withdrawal latency to 7.65±1.38 s compared to vehicle-treated animals (2.90±0.41 s; P<0.01). In animals treated with CHF3381 (30 mg/kg, p.o.) alone and (morphine 0.1 mg/kg, s.c.) alone paw withdrawal latencies did not differ significantly from vehicle-treated animals, being the respective values 3.68±0.52 and 5.17±0.86 s. Form these findings, it can be appreciated that the combination of CHF 3381 and morphine is also capable of significantly reducing allodynia after cold stimulus in the same model of chronic pain at doses which had no or modest effect on their own.

### Example 3

### Effect of CHF3381 and Morphine Combination on Mechanical Hyperalgesia in a Rat Model of Chronic Pain

The animal model of chronic pain was the same of Example 1.

Nerve injured rats developed mechanical hyperalgesia 14-21 after surgery. Indeed, in these animals the mean paw threshold was decreased to about 110 g on the ligated side compared to the contralateral side (about 350 g; P<0.01).

Mechanical hyperalgesia was assessed in neuropathic rats by using an analgesymeter according to the method reported in Randall L O et al (Arch Int Pharmacodyn Ther 1957, 111, 409-419). Mechanical nociceptive thresholds were evaluated by measuring paw withdrawal thresholds to an increasing pressure stimulus applied to the distal portion of the plantar surface of the hind paw. The site of the stimulation was on area of the hind paw between the pads at the base of the third and forth digit. A cut-off was set at 500 g to prevent any tissue damage and the endpoint was taken as a complete paw withdrawal.

The results, expressed as means ±s.e.m, are reported and discussed as follows. The level of significance was set at P<0.05.

CHF3381 30 mg/kg, p.o. alone and morphine, 0.1 mg/kg, s.c. alone had no effect on mechanical hyperalgesia in neuropathic rats. Paw withdrawal threshold values were 145.2±30.3 and 105.8±6.0 g, respectively. On the contrary, the treatment with the combination of CHF3381 30 mg/kg, p.o. and morphine 0.1 mg/kg, s.c. significantly increased the paw withdrawal threshold to 238.3±39.64 g compared to vehicle-treated animals (105.7±8.2 g; P<0.01)

The above reported findings indicate the combination of CHF 3381 and morphine is capable of significantly reducing not only allodynia but also hyperalgesia after mechanical stimulus at doses which had no effect on their own.

### Example 4

### Effect on Non-Associative Tolerance Induced by Morphine after Administration of CHF3381 in Animal Model of Inflammatory Pain

The animal model of inflammatory was a slight modification of the mouse paw formalin test originally described by Wheeler-Aceto H et al in Psychopharmacology 104, 35-44 (1991). Before formalin injection, mice were placed individually into clear plastic cylinders. After adaptation to the cage, 20 μl of 1% formalin was injected into the plantar surface of the left hind paw. Morphine was administered 30 min before formalin injection.

Mice, divided randomly into eight groups (13-16 animals/group), received treatment once daily for 4 days as follows: groups g1 and g2 received saline, 10 ml/kg i.p.; groups g3 and g4 received morphine 50 mg/kg i.p.; group g5 received morphine 50 mg/kg and CHF3381 30 mg/kg i.p.; group g6 received morphine 50 mg/kg and CHF3381 60 mg/kg i.p. On day 5, the mice received saline (g1 and g3), or morphine 6 mg/kg, i.p. (g2, g4, g5 and g6).

The treatment protocol is also reported in Table 1.

TABLE 1
Scheme of the treatment protocol

| | Treatment | Formalin test |
|---|---|---|
| | (1 × daily) | treatment |
| Group | Days 1-4 | Day 5 |
| g1 | Saline i.p. | Saline i.p. |
| g2 | Saline i.p. | Morphine 6 mg/kg i.p. |
| g3 | Morphine 50 mg/kg i.p. | Saline i.p. |
| g4 | Morphine 50 mg/kg i.p. | Morphine 6 mg/kg i.p. |
| g5 | Morphine 50 mg/kg + | Morphine 6 mg/kg i.p. |
| | CHF3381 30 mg/kg i.p. | |
| g6 | Morphine 50 mg/kg + | Morphine 6 mg/kg i.p. |
| | CHF3381 60 mg/kg i.p. | |

The amount of time, in seconds, the animals spent licking and flinching the injected paw from 10 to 40 min after formalin injection, was used as measurement of pain intensity.

The results, expressed as means ±s.e.m, are reported and discussed as follows. The level of significance was set at P<0.05.

In saline-treated mice (g1), intraplantar injection of saline at day 5 induced marked spontaneous behaviour. The licking phase measured during the late phase was 119.9±17.0. Mice receiving 6 mg/kg, i.p. of morphine at day 5 (g2) showed attenuation of basal nociception (P<0.05 vs. g1 and g3). The mean licking time was 65.9±18.3. When saline was administered i.p. at day 5, after chronic morphine, 50 mg/kg, i.p. (g3) subcutaneous injection of formalin induced marked spontaneous nociceptive behaviour (mean licking time: 143.9±19.2). Animals chronically treated with morphine 50 mg/kg, i.p. (g4) showed that spontaneous behaviour was not affected by morphine 6 mg/kg, i.p. injected at day 5. The licking time (112.1±13.4) was not significantly different from the values detected in the two control groups (g1 and g3).

When morphine 6 mg/kg, i.p. was administered at day 5, after co-administration for 4 days of CHF3381 30 mg/kg, i.p. and morphine 50 mg/kg, i.p. (g5) or with CHF3381 60 mg/kg, i.p. and morphine 50 mg/kg, i.p. (g6), its antihyperalgesic activity was significantly maintained (P<0.05 and P<0.01, respectively). The licking times were 79.5±12.5 and 70.1±8.7, respectively.

Therefore, the results demonstrate that CHF3381 administered for 4 days, together with morphine, was able to inhibit the development of non-associative tolerance induced by morphine in the formalin test. Furthermore, the tolerance was inhibited in a dose-dependent manner.

1: A medicament comprising a substance that blocks both the ion channel associated to NMDA receptors and MAO enzymes (a NMDA/MAO blocker) and a narcotic analgesic substance wherein the two substances are administrated simultaneously.

2: The medicament according to claim 1 wherein the NMDA/MAO blocker and the narcotic analgesic substance are present in a fixed combination.

3: The medicament according to claim 1 wherein the NMDA/MAO blocker is a compound represented by the general formula I: 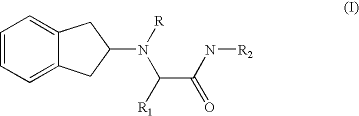
wherein:
R is hydrogen or C₁-C₄ alkyl groups;
R₁ is hydrogen, C₁-C₁₀ alkyl or optionally acylated C₁-C₄ hydroxyalkyl wherein the acylated C₁-C₄ hydroxyalkyl is selected from the group of acetyloxy C₁-C₄ alkyl, propanoyloxy C₁-C₄ alkyl, 2-methylpropanoyloxy C₁-C₄ alkyl, and benzoyloxy C₁-C₄ alkyl;
R₂ is hydrogen; C₁-C₁₀ alkyl; phenyl; phenyl C₁-C₁₀ alkyl; and pharmaceutically acceptable salts thereof.

4: The medicament according to claim 3 wherein R, R₁ and R₂ are hydrogen and the compound is N-2(indanyl)-glycinamide.

5: The medicament according to claim 1 wherein the analgesic narcotic substance is selected from the group consisting of fentanyl, hydrocodone, hydromorphone, meperidine, morphine, oxymorphone, phenyltazocine and tramadol, or pharmaceutically acceptable salts thereof such as hydrochloride, sulphate, citrate, lactate and tartrate.

6: The medicament according to claim 5 wherein the analgesic narcotic substance is morphine hydrochloride or sulphate.

7: A pharmaceutical composition comprising therapeutically effective amounts of a NMDA/MAO blocker and a narcotic analgesic substance, optionally together with at least one pharmaceutically acceptable carrier or diluent.

8: A kit comprising: a) a therapeutically effective amount of a NMDA/MAO blocker in a pharmaceutically acceptable carrier or diluent in a first unit dosage form; b) a therapeutically effective amount of a narcotic analgesic substance or a pharmaceutically acceptable salt thereof in a pharmaceutically acceptable carrier or diluent in a second unit dosage form; and c) a container for containing said first and second dosage forms in a unique packaging.

9: A method for the treatment of subjects suffering from moderate to severe pain conditions, acute or chronic, whether the pain is of neuropathic or inflammatory type or caused by different nociceptive stimuli comprising administering simultaneously a combination of a NMDA/MAO blocker and a narcotic analgesic substance.

10. The method according to claim 9 wherein the combination is a fixed combination.

11: The method according to claim 9 wherein the NMDA/MAO blocker is a compound represented by the general formula I: 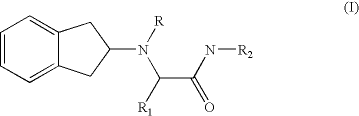
wherein:
R is hydrogen or C₁-C₄ alkyl groups;
R₁ is hydrogen, C₁-C₁₀ alkyl or optionally acylated C₁-C₄ hydroxyalkyl wherein the acylated C₁-C₄ hydroxyalkyl is selected from the group of acetyloxy C₁-C₄ alkyl, propanoyloxy C₁-C₄ alkyl, 2-methylpropanoyloxy C₁-C₄ alkyl, and benzoyloxy C₁-C₄ alkyl;
R₂ is hydrogen; C₁-C₁0 alkyl; phenyl; phenyl C1-C10 alkyl; and pharmaceutically acceptable salts thereof.

12: The method according to claim 11 wherein R, R₁ and R₂ are hydrogen and the compound is N-2(indanyl)-glycinamide.

13: The method according to claim 9 wherein the pain conditions are mixed pain conditions characterized by the presence of acute and chronic pain components.

14: The method according to claim 13 wherein the pain is a neuropathic pain refractory to the treatment with narcotic analgesic substances.
